(19) United States
(12) Patent Application Publication
(10) Pub. No.: US 2023/0212296 A1
(43) Pub. Date: Jul. 6, 2023

(54) SYSTEMS AND METHODS FOR TREATING CANCER

(71) Applicant: Arizona Board of Regents on Behalf of the University of Arizona, Tucson, AZ (US)

(72) Inventor: Noel Warfel, Tucson, AZ (US)

(21) Appl. No.: 17/928,778

(22) PCT Filed: Jun. 2, 2021
(86) PCT No.: PCT/US2021/035338

§ 371 Date: Nov. 30, 2022

Related U.S. Application Data
(60) Provisional application No. 63/033,251, filed on Jun. 2, 2020.

(51) Int. Cl. C07K 16/28; A61K 31/426; A61P 35/00

(52) U.S. Cl. CPC C07K 16/2857; A61K 31/426; A61P 35/00; C07K 2317/24; C07K 2317/30

(57) ABSTRACT

Provided herein are compositions and methods for characterizing and treating cancer. In particular, provided herein are compositions and methods for treating cancer and identifying subjects for treatment with kinase and anti-angiogenesis inhibitors.

### CROSS REFERENCE TO RELATED APPLICATION

This application claims priority to and the benefit of U.S. Provisional Application No. 63/033,251, filed Jun. 2, 2020, which is hereby incorporated by reference in its entirety.

### FIELD OF THE INVENTION

Provided herein are compositions and methods for characterizing and treating cancer. In particular, provided herein are compositions and methods for treating cancer and identifying subjects for treatment with kinase and anti-angiogenesis inhibitors.

### BACKGROUND OF THE INVENTION

The growth of a tumor to clinically malignant dimensions requires angiogenesis, the sprouting of new blood vessels from pre-existing vasculature. Not only is angiogenesis crucial for tumor growth due to oxygen and nutrient demands, it is also essential for the progression of tumor malignancy. Angiogenesis inhibitors, used either in conjunction with or in place of traditional cytotoxic chemotherapies, have shown promise in restricting tumor growth and have thus become a topic of much research.

However, patients on anti-angiogenic therapy can develop resistance either via classical resistance mechanisms, such as increased drug metabolism or an increased number of drug efflux pumps, or via compensatory release of different angiogenic inducers.

What is needed are compositions and methods for improving anti-angiogensis therapy.

### SUMMARY OF THE INVENTION

Angiogenesis is essential for sustained growth of solid tumors. HIF-1 is a master regulator of angiogenesis that is commonly activated in solid tumors. Understanding mechanisms governing the hypoxia-independent activation of HIF-1 is important for successful therapeutic targeting of tumor angiogenesis. PIM1 kinase is frequently unregulated in solid tumors and known to promote tumor growth and metastasis.

Constitutive activation of HIF-1 is frequently observed in human cancers and is associated with poor patient prognosis. Experiments described herein established a molecular mechanism responsible for the constitutive activation of HIF-1α in normoxia. PIM1 kinase directly phosphorylates HIF-1α at threonine 455, a previously uncharacterized site within its oxygen-dependent degradation domain. This phosphorylation event disrupts the ability of PHDs to bind and hydroxylate HIF-1a, interrupting its canonical degradation pathway and promoting constitutive transcription of HIF-1 target genes that drive angiogenesis. CRISPR mutants of HIF-1α T455D showed increased tumor growth, proliferation, and angiogenesis, and T455D xenograft tumors were refractory to the anti-tumor effects of PIM inhibition. These findings establish a new mechanism responsible for hypoxia-independent activation of HIF-1 and provide rationale for targeting PIM kinase as a new strategy to inhibit tumor angiogenesis.

The compositions and methods described herein provide therapueutic, diagnostic, and research uses for targeting and measuring phosphorylation of HIF-1. Such compositions and methods overcome obstacles to cancer therapy with angiogenesis and kinase inhibitors.

For example, in some embodiments, provided herein is a composition comprising: an agent (e.g., monoclonal antibody) that inhibits one or more activities of HIF-1α (e.g., by blocking the phosphorylation of HIF-1α by PIM (e.g., PIM1, PIM2, or PIM3) or binding to phosphorylated HIF-1α. In some embodiments, the phosphorylation is phosphorylation at Thr455 of HIF-1α. The present disclosure is not limited to particular monoclonal antibodies. Examples include but are not limited to, a humanized monoclonale antibody, a human monoclonal antibody, a murine monoclonal antibody, a chimeric monoclonal antibody, or a fragment of a monoclonal antibody).

In certain embodiments, the composition is a pharmaceutical composition. In some embodiments, the composition further comprises a pharmaceutically acceptable carrier. The composition may further comprise one or more additional anti-cancer agents (e.g., including but not limited to, an anti-angiogenic agent, a PIM kinase inhibitor, or a chemotherapeutic agent.

The present disclosure is not limited to particular anti-angiogenic agents. Examples include but are not limited to, axitinib, bevacizumab, cabozantinib, everolimus, lenalidomide, lenvatinib mesylate, pazopanib, ramucirumab, regorafenib, sorafenib, sunitinib, thalidomide, vandetanib, or ziv-aflibercept.

The present disclosure is not limited to particular PIM kinase inhibitors. Examples include but are not limited to, AZD1208, LGH447, SGI-1776, PIM447, SEL24, or TP-3654.

Further embodiments provide a method of treating cancer, comprising: administering a composition described herein to a subject diagnosed with cancer, wherein the administering treats one or more signs or symptoms of cancer in said subject. The present disclosure is not limited to the treatment of a specific cancer. In some embodiments, the cancer is a solid tumor (e.g., including but not limited to prostate, colon, breast, or lung).

Additional embodiments provide a method of treating cancer, comprising: a) identifying the presence of phosphorylation at Thr455 of HIF-1α in a sample from a subject diagnosed with cancer; and b) treating the subject with a PIM kinase inhibitor and/or an anti-angiogenic agent.

Also provided herein is a method of selecting a treatment for cancer, comprising: a) identifying the presence of phosphorylation at Thr455 of HIF-1α in a sample from a subject diagnosed with cancer; and selecting the subject with a PIM kinase inhibitor and/or an anti-angiogenic agent.

Any suitable method for detecting the present of phosphorylation at Thr455 of HIF-1α. For example, in some embodiments, the identifying comprises contacting the sample with a monoclonal antibody that specifically binds to said Thr455 of HIF-1α and detecting the binding.

Other embodiments provide the use of a monoclonal antibody that inhibits one or more activities of HIF-1α (e.g., by blocking the phosphorylation of HIF-1α by PIM (e.g., PIM1, PIM2, or PIM3) or binding to phosphorylated HIF-1α. to treat cancer in a subject or a monoclonal antibody that inhibits one or more activities of HIF-1α (e.g., by blocking the phosphorylation of HIF-1α by PIM (e.g., PIM1, PIM2, or PIM3) or binding to phosphorylated HIF-1α for use in treating cancer in a subject.

Additional embodiments are described herein.

### DESCRIPTION OF THE FIGURES

FIG. 1. PIM1 correlates with angiogenesis in human cancer samples. A) Representative PIM1 and CD31 staining of three human prostate cancer TMAs. B) Scoring of PIM1 and CD31 were graphed to determine the association between PIM1 and vasculature by Pearson correlation in each of the indicated TMAs. C) Association of PECAM1 and PIM1 transcript levels by Pearson correlation in TCGA prostate (PRAD), colon (COAD), and lung (LUAD) adenocarcinoma datasets.

FIG. 2. PIM1 induces angiogenesis in vivo and in vitro. A) Mice were injected with PC3-Vec or PC3-PIM1 cells and tumor volume was measured over time. B) Representative DCE-MRI trace from size-matched PC3-PIM1 and PC3-Vec tumors and C) average vascular perfusion (K^{trans}). D) Dox-PIM1 PC3 cells were transfected with siHIF1/2 prior to treatment with dox for 24 h, and lysates were collected for immunoblotting and conditioned media (CM) was harvested for tube formation assays. E) Representative images of tube formation at 1 and 6 hours after plating HUVEC cells in CM. F) Quantification of mean tube length and G) total branch points. H) VEGF-A levels in CM from the indicated conditions were measured by ELISA. I) Mice were injected with the indicated RKO cell lines, and tumor volume was measured over time. J) Vascular index was calculated by normalizing the bioluminescence signal to tumor volume. K) Tumors derived from each cell line were harvested and immunostained with PIM1, HIF-1α, CC3 and ki67. L) Quantification of IHC. *p<0.05, n.s.=not significant

FIG. 3. PIM1 is sufficient to stabilize HIF-1α and activate HIF-1 in normoxia. A) RKO colon cancer cells±PIM1 were treated with DMSO or PIM447 (1 μM) for 6 h. B) A549 and H460 lung cancer cells were stably infected with lentiviral constructs expressing Vector or PIM1. C) Dox-Vec or Dox-PIM1 PC3 cells were treated with dox for 24 h prior to DMSO or AZD1208 (3 μM) for 6 h. D) Dox-PIM1 expressing HRE-Luc were treated with Dox for 24 h prior to DMSO or AZD1208 for 6 h, and bioluminescence was measured. E and F) Dox-PIM1 cells were treated with Dox for 24 h prior to DMSO or AZD1208 for 6 h and RNA was harvested to measure the expression of hypoxia-inducible genes. F) HIF-1 target genes upregulated by 3-fold by PIM1 and reduced by AZD1208. G) Dox-PIM1 cells were treated with Dox for 24 h prior to DMSO or AZD1208 for 6 h and RNA was harvested to measure gene expression by qRT-PCR. H) RNA was harvested from the indicated cell lines and gene expression was measured by qRT-PCR. *p<0.05, n.s.=not significant

FIG. 4. PIM1 phosphorylates HIF-1α at Thr455. A) Images of Coomassie and autoradiography of in vitro kinase assays using recombinant PIM1 and HIF-1α. B) Spectra from mass spectrometry analysis of HIF-1α from in vitro kinase assay showing phosphorylation of HIF-1α at Thr455 by PIM1. C) Sequence alignment across species and schematic of HIF-1α ODDD (Thr455 highlighted in blue) D) Immunoblot of in vivo kinase assay combining recombinant PIM1 with immunoprecipitated HIF-1α constructs. E) 293T cells were transfected with WT HA-PIM1 or kinase-dead HA-PIM1-K67M and treated with DMSO or MG132 (10 pin) for 3 h. F) The indicated RKO cell lines were treated with MG-132 (10 pin) for 4 h. G) Dox-PIM1 PC3 cells were treated for 24 h with Dox and pretreated with MG-132 for 2 h followed by DMSO or PIM447 (1 μM) for 6 h; and H) A549 and H460 lung cancer cells±PIM1 were treated with MG132 for 3 h. *p<0.05.

FIG. 5. HIF1 T455 phosphorylation disrupts PHD binding and increases HIF-1α stability. A) RKO±PIM1 were incubated in hypoxia (1% O₂) for 1 h then lysed at different time-points after restoring normal oxygen (20% O₂). B) 293T cells were transfected with HA-HIF-1α, T455A or T455D and incubated in hypoxia for 4 h prior to treatment with cycloheximide (CHX, 10 μm). Densitometry was used to determine the rate of protein decay. C) 293T±PIM1 cells were transfected with HA-HIF-1α and treated with MG-132 (10 μm) (and DMSO or AZD1208 (3 μm) for 4 h. HIF-1α constructs were immunoprecipitated and ubiquitination was measured by immunoblotting and quantified by densitometry. D) A parallel ubiquitination assay was performed using 293T cells±PIM1 transfected with HA-HIF-1α, T455D or T455A. E) SW620 and PC3 cells were transfected with HA-PIM1 and lysates were collected. Relative HIF-OH (Pro564) is graphed. F) 293T-PIM1 cells were transfected with HA-HIF-1α, T455D or T455A. HA-HIF-1α constructs were immunoprecipitated and blotted for HIF-OH (Pro564). The ratio of hydroxylated to total HIF-1α is graphed. G) 293T±PIM1 cells were transfected with HA-HIF-1α and treated with DMSO or PIM447 (3 μm) for 4 h. HA-HIF1α constructs were immunoprecipitated and PHD2 was probed by western blotting; relative abundance was calculated by densitometry. H) 293T cells were transfected with HA-HIF-1α, T455A or T445D. HA-HIF1α constructs were immunoprecipitated and PHD2 was probed by western blotting; relative abundance quantified below *p<0.05, n.s.=not significant.

FIG. 6. HIF-1α-T455D CRISPR mutants increase tumor growth and are resistant to PIM inhibition. A) The indicated number of SW620, B24, and C34 cells were plated and allowed to grow for 48 h before analysis by MTT. B) Twenty thousand SW620, B24 and C34 cells were treated with various concentrations AZD1208 or PIM447 for 24 h before analysis by MTT. C) SW620, B24, and C34 were treated with PIM447 (3 μm) for 6 h. D) Representative images of tube formation after 24 h incubation in the indicated CM. E) Mean tube length and F) total branch points were quantified. G) Mice were injected 5×10⁶ SW620 or C34 cells, treated with vehicle or AZD1208 (30 mg/kg), and tumor volume was measured over time. H) Tumors from each cohort were stained for H&E, PIM1, ki67, HIF-1α and CC3. I and J) Quantification of HIF-1α and CC3 staining in each cohort. K) RNA was harvested from tumor tissue from each cohort and mRNA expression was measured by qRT-PCR. L) Model depicting the mechanism and outcome of PIM1-mediated phosphorylation of HIF1α.

FIG. 7. Sequence of HIF1α (SEQ ID NO:1).

### DEFINITIONS

To facilitate an understanding of the present invention, a number of terms and phrases are defined below:

The expression “amino acid position corresponding to” a position in a reference sequence and similar expression is intended to identify the amino acid residue that in the primary or spatial structure corresponds to the particular position in the reference sequence. This can be done by aligning a given sequence with the reference sequence and identifying the amino acid residue that aligns with the particular position in the reference sequence.

The term “sample” as used herein is used in its broadest sense. In one sense it can refer to a tissue sample. In another sense, it is meant to include a specimen or culture obtained from any source, as well as biological. Biological samples may be obtained from animals (including humans) and encompass fluids, solids, tissues, and gases. Biological samples include, but are not limited to blood products, such as plasma, serum and the like. These examples are not to be construed as limiting the sample types applicable to the present disclosure.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries.

As used herein “immunoglobulin” refers to any class of structurally related proteins in the serum and the cells of the immune system that function as antibodies. In some embodiments, an immunoglobulin is the distinct antibody molecule secreted by a clonal line of B cells.

As used herein, the term “antibody” refers to a whole antibody molecule or a fragment thereof (e.g., fragments such as Fab, Fab′, and F(ab′)2), it may be a polyclonal or monoclonal antibody, a chimeric antibody, a humanized antibody, a human antibody, etc. A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa).

In a native antibody, a heavy chain comprises a variable region, V_{H}, and three constant regions, C_{H1}, C_{H2}, and C_{H3}. The V_{H} domain is at the amino-terminus of the heavy chain, and the C_{H3} domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. H3, for example, in certain instances, can be as short as two amino acid residues or greater than 26. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol. 196:901-917; or Chothia, C. et al. Nature 342:878-883 (1989). In the present application, the term “CDR” refers to a CDR from either the light or heavy chain, unless otherwise specified.

As used herein, the term “heavy chain” refers to a polypeptide comprising sufficient heavy chain variable region sequence to confer antigen specificity either alone or in combination with a light chain.

As used herein, the term “light chain” refers to a polypeptide comprising sufficient light chain variable region sequence to confer antigen specificity either alone or in combination with a heavy chain.

As used herein, when an antibody or other entity “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “an antibody that blocks phosphorylation of HIF1α” refers to an antibody which specifically blocks the phosphorylation of HIF1α by a PIM kinase (e.g., by binding to a specific phosphorylation site on HIF1α).

As used herein, the term “monoclonal antibody” refers to an antibody which is a member of a substantially homogeneous population of antibodies that specifically bind to the same epitope. In certain embodiments, a monoclonal antibody is secreted by a hybridoma. In certain such embodiments, a hybridoma is produced according to certain methods; See, e.g., Kohler and Milstein (1975) Nature 256: 495-499; herein incorporated by reference in its entirety. In certain embodiments, a monoclonal antibody is produced using recombinant DNA methods (see, e.g., U.S. Pat. No. 4,816,567). In certain embodiments, a monoclonal antibody refers to an antibody fragment isolated from a phage display library. See, e.g., Clackson et al. (1991) Nature 352: 624-628; and Marks et al. (1991) J. Mol. Biol. 222: 581-597; herein incorporated by reference in their entireties.

The modifying word “monoclonal” indicates properties of antibodies obtained from a substantially-homogeneous population of antibodies, and does not limit a method of producing antibodies to a specific method. For various other monoclonal antibody production techniques, see, e.g., Harlow and Lane (1988) Antibodies: A Laboratory Manual (Cold Spring Harbor Laboratory, Cold Spring Harbor, N.Y.); herein incorporated by reference in its entirety.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab” fragment comprises one light chain and the Cm and variable region of one heavy chain. The heavy chain of a Fab molecule cannot form a disulfide bond with another heavy chain molecule. A “Fab′” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule.

An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen.

As used herein, the term “chimeric antibody” refers to an antibody made up of components from at least two different sources. In certain embodiments, a chimeric antibody comprises a portion of an antibody derived from a first species fused to another molecule, e.g., a portion of an antibody derived from a second species. In certain such embodiments, a chimeric antibody comprises a portion of an antibody derived from a non-human animal fused to a portion of an antibody derived from a human. In certain such embodiments, a chimeric antibody comprises all or a portion of a variable region of an antibody derived from a non-human animal fused to a constant region of an antibody derived from a human.

As used herein, the term “natural antibody” refers to an antibody in which the heavy and light chains of the antibody have been made and paired by the immune system of a multicellular organism. For example, the antibodies produced by the antibody-producing cells isolated from a first animal immunized with an antigen are natural antibodies. Natural antibodies contain naturally-paired heavy and light chains. The term “natural human antibody” refers to an antibody in which the heavy and light chains of the antibody have been made and paired by the immune system of a human subject.

Native human light chains are typically classified as kappa and lambda light chains. Native human heavy chains are typically classified as mu, delta, gamma, alpha, or epsilon, and define the antibody's isotype as IgM, IgD, IgG, IgA, and IgE, respectively. IgG has subclasses, including, but not limited to, IgG1, IgG2, IgG3, and IgG4. IgM has subclasses including, but not limited to, IgM1 and IgM2. IgA has subclasses including, but not limited to, IgA1 and IgA2. Within native human light and heavy chains, the variable and constant regions are typically joined by a “J” region of about 12 or more amino acids, with the heavy chain also including a “D” region of about 10 more amino acids. See, e.g., Fundamental Immunology (1989) Ch. 7 (Paul, W., ed., 2nd ed. Raven Press, N.Y.); herein incorporated by reference in its entirety.

The term “antigen-binding site” refers to a portion of an antibody capable of specifically binding an antigen. In certain embodiments, an antigen-binding site is provided by one or more antibody variable regions.

The term “epitope” refers to any polypeptide determinant capable of specifically binding to an immunoglobulin or a T-cell or B-cell receptor. In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three dimensional structural characteristics (e.g., a “conformational” epitope) and/or specific charge characteristics.

As used herein, the term “multivalent”, particularly when used in describing an agent that is an antibody, antibody fragment, or other binding agent, refers to the presence of two or more (e.g., 2, 3, 4, 5, 6, 7, 8, 9, 10, or more) antigen binding sites on the agent.

As used herein, the term “multispecific”, particularly when used in describing an agent that is an antibody, antibody fragment, or other binding agent, refers to the capacity to of the agent to bind two or more (e.g., 2, 3, 4, 5, 6, 7, 8, 9, 10, or more) targets (e.g., unrelated targets). For example, a bispecific antibody recognizes and binds to two different antigens.

An epitope is defined as “the same” as another epitope if a particular antibody specifically binds to both epitopes. In certain embodiments, polypeptides having different primary amino acid sequences may comprise epitopes that are the same. In certain embodiments, epitopes that are the same may have different primary amino acid sequences. Different antibodies are said to bind to the same epitope if they compete for specific binding to that epitope.

A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues, which are typically incorporated by chemical peptide synthesis rather than by synthesis in biological systems. These include, but are not limited to, peptidomimetics and other reversed or inverted forms of amino acid moieties. Naturally occurring residues may be divided into classes based on common side chain properties, for example: hydrophobic: norleucine, Met, Ala, Val, Leu, and Ile; neutral hydrophilic: Cys, Ser, Thr, Asn, and Gln; acidic: Asp and Glu; basic: His, Lys, and Arg; residues that influence chain orientation: Gly and Pro; and aromatic: Trp, Tyr, and Phe. Non-conservative substitutions may involve the exchange of a member of one of these classes for a member from another class; whereas conservative substitutions may involve the exchange of a member of one of these classes for another member of that same class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families (see above). The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “selectively” (e.g., as in “selectively targets,” “selectively binds,” etc.) refers to the preferential association of an agent (e.g., antibody or antibody fragment) for a particular entity (e.g., antigen, antigen presenting cell, etc.). For example, an agent selectively targets a particular cell population if it preferentially associates (e.g., binds an epitope or set of epitopes presented thereon) with that cell population over another cell population (e.g., all other cell populations present in a sample). The preferential association may be by a factor of at least 2, 4, 6, 8, 10, 20, 50, 100, 10³, 10⁴, 10⁵, 10⁶, or more, or ranges there between. An agent that X-fold selectively targets a particular cell populations, associates with that cell population by at least X-fold more than other cell populations present.

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., humans).

The term “diagnosed,” as used herein, refers to the recognition of a disease by its signs and symptoms, or genetic analysis, pathological analysis, histological analysis, and the like.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded) but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues (e.g., biopsy samples), cells, and gases. Biological samples include blood products, such as plasma, serum and the like. Such examples are not however to be construed as limiting the sample types applicable to the present invention.

### DETAILED DESCRIPTION OF THE INVENTION

Angiogenesis, or the branching of blood vessels, is a rate-limiting step in the development of solid tumors (Folkman, 1971 N Engl J Med 285, 1182-1186). Without angiogenesis, solid tumors cannot sustain proliferation due to a lack of oxygen and nutrients (Muthukkaruppan et al., 1982 J Natl Cancer Inst 69, 699-708). Hypoxia-inducible factor 1 (HIF-1) is a basic helix-loop-helix-PAS domain transcription factor that is a critical mediator of the cellular response to oxygen deprivation and a key driver of tumor angiogenesis (Semenza, 1999 Cell 98, 281-284). HIF-1 is a heterodimer that consists of a constitutively expressed subunit, HIF-1β, and HIF-1α, a subunit whose expression is tightly regulated in an oxygen-dependent manner. In the presence of oxygen, cytoplasmic HIF-1α is rapidly hydroxylated on prolines 402 (Pro402) and 564 (Pro564), located within its oxygen-dependent degradation domain (ODDD), by prolyl hydroxylase-domain proteins (PHDs) 1-3 (Bruick and McKnight, 2001 Science 294, 1337-1340; Semenza, 2004 Physiology 19, 176-182). Hydroxylation causes the von Hippel-Lindau (VHL) tumor suppressor protein to recognize HIF-1α and recruit a ubiquitin-protein ligase complex that leads to the ubiquitination and rapid degradation of HIF-1α by the 26S proteasome (Maxwell et al., 1999 Nature 399, 271-275). In the absence of oxygen, PHDs become inactive and no longer hydroxylate HIF-1α, allowing it to accumulate in the cell and translocate to the nucleus, where it binds to hypoxia-response elements (HREs) to promote the transcription of target genes, particularly pro-angiogenic factors (Ivan et al., 2001 Science 292, 464-468). Therefore, understanding the mechanism by which tumor cells sustain HIF-1α expression is critical for understanding solid tumor growth and identifying new and effective ways to target angiogenesis therapeutically.

Constitutive expression of HIF-1α is common in human cancers, regardless of oxygen tension. Stabilization of HIF-1α in normoxia has been attributed to genetic alterations, such as loss of VHL, as well as transcriptional upregulation due to the activation of oncogenic signaling pathways, such as NF-κB, STAT3, and Sp1. HIF-1α mRNA and protein synthesis are also induced by oncogenic signaling pathways, including PI3K and RAS (Baldewijns et al., 2010 J Pathol 221, 125-138; Hua Zhong, 2000 60, 1541-1545; Richard D E, 1999 J Biol Chem 274, 32631-32637). Post-translational modification also plays a critical role in controlling HIF-1α expression and function. Direct phosphorylation by ERK blocks the nuclear export of HIF-1a and promotes its accumulation in the nucleus, resulting in higher transcriptional activation (Mylonis et al., 2006 J Biol Chem 281, 33095-33106). It has also been reported that phosphorylation of HIF-1α by various kinases can control its protein stability. Phosphorylation by glycogen synthase kinase 3 and Polo-like kinase 3 promotes HIF-1α degradation (Flegel D, 2007 Mol Cell Biol 27, 3253-3265; Isaacs et al., 2002 J Biol Chem 277, 29936-29944; Xu D, 2010 J Biol Chem, 38944-38950), whereas phosphorylation by CDK1, ATM, and PKA have been reported to stabilize HIF-1α (Bullen et al., 2016 Sci Signal 9, ra56; Cam et al., 2010 Mol Cell 40, 509-520; Warfel et al., 2013 Cell cycle 12, 3689-3701). Regardless of the mechanism, stabilization of HIF-1α in normoxia results in the constitutive upregulation of genes that initiate and sustain angiogenesis during tumor growth (Hartwich et al., 2013 Journal of pediatric surgery 48, 39-46). Hence, the identification of oxygen-independent mechanisms that regulate HIF-1a expression is extremely valuable in the effort to understand tumor progression as well as to effectively target HIF-1 as a therapeutic strategy.

The Proviral integration site for Moloney murine leukemia virus (PIM) kinases are a family of serine-threonine kinases that are known to promote tumorigenesis by impacting cell cycle progression, survival, and proliferation (Nawijn et al., 2011 Nature reviews Cancer 11, 23-34). Pim1 expression is elevated in ˜50% of human prostate cancer specimens, particularly in high Gleason grade and aggressive metastatic prostate cancer cases, highlighting its ability to enhance tumorigenesis (Chen et al., 2005 MCR 3, 443-451; Dhanasekaran et al., 2001 Nature 412, 822-826; Xie Y, 2006 Oncogene; Horiuchi et al., 2016 Nature medicine 22, 1321-1329). The Pim family is also elevated in a host of other solid tumors, including colon, breast, and lung cancer, with overexpression leading to higher staging, increased metastasis, and diminished overall survival. (Braso-Maristany et al., 2017 Erratum: PIM1 kinase; Chauhan et al., 2020 Oncogene 39, 2597-2611; Dhanasekaran et al., 2001 Nature 412, 822-826; Gao et al., 2019 Breast Cancer 26, 663-671; Xie Y, 2006 Oncogene; Zhang et al., 2018 Cancer Sci 109, 1468-1479). As a result, several small molecule PIM kinase inhibitors are actively being tested against hematological and solid tumors in clinical trials (NCT03715504 and NCT03008187). Experiments conducted during the course of development of embodiments of the present disclosure established expression of PIM1 as an important factor responsible for driving tumor angiogenesis. PIM1 promotes angiogenesis through a signaling axis directly linking PIM1 to HIF-1 via a previously uncharacterized direct phosphorylation event that disrupts the canonical HIF-1α degradation pathway. The results indicate that the ability of PIM1 to induce angiogenesis and tumor growth is dependent on stabilization of HIF-1 and that the anti-tumor effects of PIM inhibitors are largely due to their anti-angiogenic properties.

Accordingly, in some embodiments, the present disclosure provides research, screening, and therapeutic methods that monitor or target the phosphorylation of HIF-1α.

### I. Agents

In some embodiments, the present disclosure provides agents that inhibit one or more activities of HIF-1α (e.g., that specifically bind to, identify, and/or block phosphorylation of HIF-1α). In some embodiments, the agent is an antibody or immunoglobulin.

In some embodiments, the immunoglobulin molecule is composed of two identical heavy and two identical light polypeptide chains, held together by interchain disulfide bonds. Each individual light and heavy chain folds into regions of about 110 amino acids, assuming a conserved three-dimensional conformation. The light chain comprises one variable region (termed VL) and one constant region (CL), while the heavy chain comprises one variable region (VH) and three constant regions (CH1, CH2 and CH3). Pairs of regions associate to form discrete structures. In particular, the light and heavy chain variable regions, VL and VH, associate to form an “FV” area that contains the antigen-binding site.

The variable regions of both heavy and light chains show considerable variability in structure and amino acid composition from one antibody molecule to another, whereas the constant regions show little variability. Each antibody recognizes and binds an antigen through the binding site defined by the association of the heavy and light chain, variable regions into an FV area. The light-chain variable region VL and the heavy-chain variable region VH of a particular antibody molecule have specific amino acid sequences that allow the antigen-binding site to assume a conformation that binds to the antigen epitope recognized by that particular antibody.

Within the variable regions are found regions in which the amino acid sequence is extremely variable from one antibody to another. Three of these so-called “hypervariable” regions or “complementarity-determining regions” (CDR's) are found in each of the light and heavy chains. The three CDRs from a light chain and the three CDRs from a corresponding heavy chain form the antigen-binding site.

The amino acid sequences of many immunoglobulin heavy and light chains have been determined and reveal two important features of antibody molecules. First, each chain consists of a series of similar, although not identical, sequences, each about 110 amino acids long. Each of these repeats corresponds to a discrete, compactly folded region of protein structure known as a protein domain. The light chain is made up of two such immunoglobulin domains, whereas the heavy chain of the IgG antibody contains four.

The second important feature revealed by comparisons of amino acid sequences is that the amino-terminal sequences of both the heavy and light chains vary greatly between different antibodies. The variability in sequence is limited to approximately the first 110 amino acids, corresponding to the first domain, whereas the remaining domains are constant between immunoglobulin chains of the same isotype. The amino-terminal variable or V domains of the heavy and light chains (V_{H} and V_{L}, respectively) together make up the V region of the antibody and confer on it the ability to bind specific antigen, while the constant domains (C domains) of the heavy and light chains (C_{H} and C_{L}, respectively) make up the C region. The multiple heavy-chain C domains are numbered from the amino-terminal end to the carboxy terminus, for example C_{H}1, C_{H}2, and so on.

The protein domains described above associate to form larger globular domains. Thus, when fully folded and assembled, an antibody molecule comprises three relatively equal-sized globular portions joined by a flexible stretch of polypeptide chain known as the hinge region. Each arm of this Y-shaped structure is formed by the association of a light chain with the amino-terminal half of a heavy chain, whereas the trunk of the Y is formed by the pairing of the carboxy-terminal halves of the two heavy chains. The association of the heavy and light chains is such that the V_{H} and V_{L} domains are paired, as are the C_{H}1 and C_{L} domains. The C_{H}3 domains pair with each other but the C_{H}2 domains do not interact; carbohydrate side chains attached to the C_{H}2 domains lie between the two heavy chains. The two antigen-binding sites are formed by the paired V_{H} and V_{L} domains at the ends of the two arms of the Y.

Proteolytic enzymes (proteases) that cleave polypeptide sequences have been used to dissect the structure of antibody molecules and to determine which parts of the molecule are responsible for its various functions. Limited digestion with the protease papain cleaves antibody molecules into three fragments. Two fragments are identical and contain the antigen-binding activity. These are termed the Fab fragments, for Fragment antigen binding. The Fab fragments correspond to the two identical arms of the antibody molecule, which contain the complete light chains paired with the V_{H} and C_{H}1 domains of the heavy chains. The other fragment contains no antigen-binding activity but was originally observed to crystallize readily, and for this reason was named the Fc fragment, for Fragment crystallizable. This fragment corresponds to the paired C_{H}2 and C_{H}3 domains and is the part of the antibody molecule that interacts with effector molecules and cells. The functional differences between heavy-chain isotypes lie mainly in the Fc fragment. The hinge region that links the Fc and Fab portions of the antibody molecule is in reality a flexible tether, allowing independent movement of the two Fab arms, rather than a rigid hinge.

In some embodiments, provided herein is an antibody that specifically binds to HIF-1α phosphorylated at Thr455. In some embodiments, provided herein is an antibody that blocks the phosphorylation of HIF-1α at Thr455.

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

In some embodiments, the antibody is a chimeric antibody. In some embodiments, chimeras comprise constant region sequences from a different species or isotype as described herein. In some embodiments, the antibody is a fragment (e.g., a fragment that retains binding to the target epitope).

Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

Further provided is an isolated polynucleotide encoding a heavy and/or light chain variable region of the antibody described herein. Such nucleic acids find use in production and/or screening of antibodies.

Addition embodiments provide a vector comprising a polynucleotide as described herein, a recombinant host cell comprising a polynucleotide as described herein, or a recombinant host cell comprising a vector as described herein.

The disclosure also features methods for producing any of the antibodies or antigen-binding fragments thereof described herein. In some embodiments, methods for preparing an antibody described herein can include immunizing a subject (e.g., a non-human mammal) with an appropriate immunogen. For example, to generate an antibody that binds to HIF-1α phosphorylated at Thr455 or blocks the phosphorylation, one can immunize a suitable subject (e.g., a non-human mammal such as a rat, a mouse, a gerbil, a hamster, a dog, a cat, a pig, a goat, a horse, or a non-human primate) with a full-length or fragment of a HIF-1a polypeptide (e.g., phosphorylated at Thr455).

A suitable subject (e.g., a non-human mammal) can be immunized with the appropriate antigen along with subsequent booster immunizations a number of times sufficient to elicit the production of an antibody by the mammal. The immunogen can be administered to a subject (e.g., a non-human mammal) with an adjuvant. Adjuvants useful in producing an antibody in a subject include, but are not limited to, protein adjuvants; bacterial adjuvants, e.g., whole bacteria (BCG, Corynebacterium parvum or Salmonella minnesota) and bacterial components including cell wall skeleton, trehalose dimycolate, monophosphoryl lipid A, methanol extractable residue (MER) of tubercle bacillus, complete or incomplete Freund's adjuvant; viral adjuvants; chemical adjuvants, e.g., aluminum hydroxide, and iodoacetate and cholesteryl hemisuccinate. Other adjuvants that can be used in the methods for inducing an immune response include, e.g., cholera toxin and parapoxvirus proteins. See also Bieg et al. (1999) Autoimmunity 31(1):15-24. See also, e.g., Lodmell et al. (2000) Vaccine 18:1059-1066; Johnson et al. (1999) J Med Chem 42:4640-4649; Baldridge et al. (1999) Methods 19:103-107; and Gupta et al. (1995) Vaccine 13(14): 1263-1276.

In some embodiments, the methods include preparing a hybridoma cell line that secretes a monoclonal antibody that binds to the immunogen. For example, a suitable mammal such as a laboratory mouse is immunized with a polypeptide as described above. Antibody-producing cells (e.g., B cells of the spleen) of the immunized mammal can be isolated two to four days after at least one booster immunization of the immunogen and then grown briefly in culture before fusion with cells of a suitable myeloma cell line. The cells can be fused in the presence of a fusion promoter such as, e.g., vaccinia virus or polyethylene glycol. The hybrid cells obtained in the fusion are cloned, and cell clones secreting the desired antibodies are selected. For example, spleen cells of Balb/c mice immunized with a suitable immunogen can be fused with cells of the myeloma cell line PAI or the myeloma cell line Sp2/0-Ag 14. After the fusion, the cells are expanded in suitable culture medium, which is supplemented with a selection medium, for example HAT medium, at regular intervals in order to prevent normal myeloma cells from overgrowing the desired hybridoma cells. The obtained hybridoma cells are then screened for secretion of the desired antibodies, e.g., an antibody that binds to canine N-cadherin.

In some embodiments, a suitable antibody is identified from a non-immune biased library as described in, e.g., U.S. Pat. No. 6,300,064 (to Knappik et al.; Morphosys AG) and Schoonbroodt et al. (2005) Nucleic Acids Res 33(9):e81.

In some embodiments, the methods described herein can involve, or be used in conjunction with, e.g., phage display technologies, bacterial display, yeast surface display, eukaryotic viral display, mammalian cell display, and cell-free (e.g., ribosomal display) antibody screening techniques (see, e.g., Etz et al. (2001) J Bacteriol 183:6924-6935; Cornelis (2000) Curr Opin Biotechnol 11:450-454; Klemm et al. (2000) Microbiology 146:3025-3032; Kieke et al. (1997) Protein Eng 10:1303-1310; Yeung et al. (2002) Biotechnol Prog 18:212-220; Boder et al. (2000) Methods Enzymology 328:430-444; Grabherr et al. (2001) Comb Chem High Throughput Screen 4:185-192; Michael et al. (1995) Gene Ther 2:660-668; Pereboev et al. (2001) J Virol 75:7107-7113; Schaffitzel et al. (1999) J Immunol Methods 231:119-135; and Hanes et al. (2000) Nat Biotechnol 18:1287-1292).

In phage display methods, functional antibody domains are displayed on the surface of phage particles which carry the polynucleotide sequences encoding them. Such phage can be utilized to display antigen-binding domains of antibodies, such as Fab, Fv, or disulfide-bond stabilized Fv antibody fragments, expressed from a repertoire or combinatorial antibody library (e.g., human or murine). Phage used in these methods are typically filamentous phage such as fd and M13. The antigen binding domains are expressed as a recombinantly-fused protein to any of the phage coat proteins pIII, pVIII, or pIX. See, e.g., Shi et al. (2010) JMB 397:385-396. Examples of phage display methods that can be used to make the immunoglobulins, or fragments thereof, described herein include those disclosed in Brinkman et al. (1995) J Immunol Methods 182:41-50; Ames et al. (1995) J Immunol Methods 184:177-186; Kettleborough et al. (1994) Eur J Immunol 24:952-958; Persic et al. (1997) Gene 187:9-18; Burton et al. (1994) Advances in Immunology 57:191-280; and PCT publication nos. WO 90/02809, WO 91/10737, WO 92/01047, WO 92/18619, WO 93/11236, WO 95/15982, and WO 95/20401. Suitable methods are also described in, e.g., U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

In some embodiments, the phage display antibody libraries can be generated using mRNA collected from B cells from the immunized mammals. For example, a splenic cell sample comprising B cells can be isolated from mice immunized with a polypeptide as described above. mRNA can be isolated from the cells and converted to cDNA using standard molecular biology techniques. See, e.g., Sambrook et al. (1989) “Molecular Cloning: A Laboratory Manual, 2.sup.nd Edition,” Cold Spring Harbor Laboratory Press, Cold Spring Harbor, N.Y.; Harlow and Lane (1988), supra; Benny K. C. Lo (2004), supra; and Borrebaek (1995), supra. The cDNA coding for the variable regions of the heavy chain and light chain polypeptides of immunoglobulins are used to construct the phage display library. Methods for generating such a library are described in, e.g., Merz et al. (1995) J Neurosci Methods 62(1-2):213-9; Di Niro et al. (2005) Biochem J 388(Pt 3):889-894; and Engberg et al. (1995) Methods Mol Biol 51:355-376.

In some embodiments, a combination of selection and screening can be employed to identify an antibody of interest from, e.g., a population of hybridoma-derived antibodies or a phage display antibody library. Suitable methods are known in the art and are described in, e.g., Hoogenboom (1997) Trends in Biotechnology 15:62-70; Brinkman et al. (1995), supra; Ames et al. (1995), supra; Kettleborough et al. (1994), supra; Persic et al. (1997), supra; and Burton et al. (1994), supra. For example, a plurality of phagemid vectors, each encoding a fusion protein of a bacteriophage coat protein (e.g., pIII, pVIII, or pIX of M13 phage) and a different antigen-combining region are produced using standard molecular biology techniques and then introduced into a population of bacteria (e.g., E. coli). Expression of the bacteriophage in bacteria can, in some embodiments, require use of a helper phage. In some embodiments, no helper phage is required (see, e.g., Chasteen et al., (2006) Nucleic Acids Res 34(21):e145). Phage produced from the bacteria are recovered and then contacted to, e.g., a target antigen bound to a solid support (immobilized). Phage may also be contacted to antigen in solution, and the complex is subsequently bound to a solid support.

A subpopulation of antibodies screened using the above methods can be characterized for their specificity and binding affinity for a particular antigen using any immunological or biochemical based method. For example, specific binding of an antibody to canine N-cadherin, may be determined for example using immunological or biochemical based methods such as, but not limited to, an ELISA assay, SPR assays, immunoprecipitation assay, affinity chromatography, and equilibrium dialysis as described above. Immunoassays which can be used to analyze immunospecific binding and cross-reactivity of the antibodies include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays.

In embodiments where the selected CDR amino acid sequences are short sequences (e.g., fewer than 10-15 amino acids in length), nucleic acids encoding the CDRs can be chemically synthesized as described in, e.g., Shiraishi et al. (2007) Nucleic Acids Symposium Series 51(1):129-130 and U.S. Pat. No. 6,995,259. For a given nucleic acid sequence encoding an acceptor antibody, the region of the nucleic acid sequence encoding the CDRs can be replaced with the chemically synthesized nucleic acids using standard molecular biology techniques. The 5′ and 3′ ends of the chemically synthesized nucleic acids can be synthesized to comprise sticky end restriction enzyme sites for use in cloning the nucleic acids into the nucleic acid encoding the variable region of the donor antibody. Alternatively, fragments of chemically synthesized nucleic acids, together capable of encoding an antibody, can be joined together using DNA assembly techniques.

The antibodies or antigen-binding fragments thereof described herein can be produced using a variety of techniques in the art of molecular biology and protein chemistry. For example, a nucleic acid encoding one or both of the heavy and light chain polypeptides of an antibody can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), cytomegalovirus, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, viral infection, dextran-mediated transfection, polybrene-mediated transfection, protoplast fusion, and direct microinjection.

Appropriate host cells for the expression of antibodies or antigen-binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof are expressed in, and purified from, transgenic animals (e.g., transgenic mammals). For example, an antibody is produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression will vary with the choice of the expression vector and the host cell. For example, antibodies expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors, and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the antibodies and fragments thereof can be isolated. An antibody or fragment thereof can be isolated or purified in a variety of ways depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3.sup.rd edition,” Springer-Verlag, New York City, N.Y. The degree of purification necessary will vary depending on the desired use. In some instances, no purification of the expressed antibody or fragments thereof will be necessary.

Methods for determining the yield or purity of a purified antibody or fragment thereof are include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

In some examples, the antibodies to an epitope for an interested protein as described herein or a fragment thereof are humanized antibodies. Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al. 1986. Nature 321:522-525; Riechmann et al. 1988. Nature 332:323-329; Presta. 1992. Curr. Op. Struct. Biol. 2:593-596). Humanization can be essentially performed following methods of Winter and co-workers (see, e.g., Jones et al. 1986. Nature 321:522-525; Riechmann et al. 1988. Nature 332:323-327; and Verhoeyen et al. 1988. Science 239:1534-1536), by substituting rodent CDRs or CDR sequences for the corresponding sequences of a human antibody. Accordingly, such humanized antibodies are chimeric antibodies (e.g., U.S. Pat. No. 4,816,567), wherein substantially less than an intact human variable domain has been substituted by the corresponding sequence from a non-human species.

In various examples the antibodies to an epitope of an interested protein as described herein or a fragment thereof are human antibodies. Human antibodies can also be produced using various techniques known in the art, including phage display libraries (Hoogenboom and Winter. 1991. J. Mol. Biol. 227:381-388; Marks et al. 1991. J. Mol. Biol. 222:581-597) or the preparation of human monoclonal antibodies [e.g., Cole et al. 1985. Monoclonal Antibodies and Cancer Therapy Liss; Boerner et al. 1991. J. Immunol. 147(486-95]. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in most respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al. 1992. Bio/Technology 10:779-783; Lonberg et al. 1994. Nature 368:856-859; Morrison. 1994. Nature 368:812-13; Fishwild et al. 1996. Nature Biotechnology 14:845-51; Neuberger. 1996. Nature Biotechnology 14:826; Lonberg and Huszar. 1995. Intern. Rev. Immunol. 13:65-93. U.S. Pat. No. 6,719,971 also provides guidance to methods of generating humanized antibodies.

Any of these compositions, alone or in combination with other compositions of the present disclosure, may be provided in the form of a kit. In some embodiments, antibodies and reagents are provided in one or more containers. Kits may further comprise appropriate controls and/or detection reagents. For example, in some embodiments, kits comprise one or more of a multiwell plate, lateral flow strips, beads, analysis software, and the like.

Embodiments of the present invention further provide pharmaceutical compositions (e.g., comprising one or more of the therapeutic agents described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the active agents of the formulation.

Dosing is dependent on severity and responsiveness of the disease state or condition to be treated, with the course of treatment lasting from several days to several months, or until a cure is effected or a diminution of the disease state is achieved. In some embodiments, treatment is administered in one or more courses, where each course comprises one or more doses per day for several days (e.g., 1, 2, 3, 4, 5, 6) or weeks (e.g., 1, 2, or 3 weeks, etc.). In some embodiments, courses of treatment are administered sequentially (e.g., without a break between courses), while in other embodiments, a break of 1 or more days, weeks, or months is provided between courses. In some embodiments, treatment is provided on an ongoing or maintenance basis (e.g., multiple courses provided with or without breaks for an indefinite time period). Optimal dosing schedules can be calculated from measurements of drug accumulation in the body of the patient. The administering physician can readily determine optimum dosages, dosing methodologies and repetition rates.

In some embodiments, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues.

### II. Therapeutic Methods

In some embodiments, compounds described herein find use in the treatment of cancer. The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include solid tumors, soft tissue tumors, and metastases thereof. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non small cell carcinoma of the lung, and cancer of the small intestine. Other exemplary cancers include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primacy; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; TCell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated or prevented in accordance with the methods described herein.

For example, in some embodiments, provided herein is a method of treating cancer, comprising: administering a monoclonal antibody that inhibits one or more activities of HIF-1a. In some embodiments, the antibody blocks the phosphorylation of HIF-1α by PIM (e.g., at Thr 455) and/or binds to phosphorylated HIF-1α. Exemplary antibodies are described above.

Additional embodiments provide a method of identifying subjects for treatment with a PIM kinase inhibitor and/or an anti-angiogenic agent, comprising identifying the presence of phosphorylation at Thr455 of HIF-1α in a sample from a subject diagnosed with cancer. Such individuals are identified as candidates for treatment with a PIM kinase inhibitor and/or an anti-angiogenic agent. In some embodiments, one or more of a PIM kinase inhibitor and/or an anti-angiogenic agent are administered to such subjects. In some embodiments, subjects that lack phosphorylation at Thr455 of HIF-1α are not administered a PIM kinase inhibitor and/or an anti-angiogenic agent.

The present disclosure is not limited to particular anti-angiogenic agents. Examples include but are not limited to, axitinib, bevacizumab, cabozantinib, everolimus, lenalidomide, lenvatinib mesylate, pazopanib, ramucirumab, regorafenib, sorafenib, sunitinib, thalidomide, vandetanib, or ziv-aflibercept.

The present disclosure is not limited to particular PIM kinase inhibitors. Examples include but are not limited to, AZD1208, LGH447, SGI-1776, PIM447, SEL24, or TP-3654.

The presence of phosphorylation at Thr455 of HIF-1α is identified using any suitable method (e.g., a phosphorylation specific antibody described herein).

In some embodiments, a computer-based analysis program is used to translate the raw data generated by the detection assay (e.g., the presence or absence of phosphorylation at Thr455 of HIF-1α) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., presence or absence of phosphorylation at Thr455 of HIF-1α), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., phosphorylation at Thr455 of HIF-1α) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

### EXPERIMENTAL

The following examples are provided in order to demonstrate and further illustrate certain preferred embodiments and aspects of the present invention and are not to be construed as limiting the scope thereof.

### Example 1

### Example 1

### Methods

### Cell Lines

Parental and genetically modified A549, H460, HEK293T, RKO, and SW620 cells were maintained in DMEM medium containing 10% FBS. SW620 HIF-1α-T455D clone B24 and clone C34 cell lines were generated by CRISPR-cas9 mediated mutagenesis using ssODN AAAATTACAGAATATAAATTTGGCAATGTCTCCATTACCCGATGCTGAAACGCCA AAGCCACTTCGAAGTAGTGCTGACCCTG (SEQ ID NO:2). PC3/VEC and PC3/PIM1 cell lines were maintained in RPMI medium containing 10% FBS. Human umbilical vein endothelial cells (HUVECs) from Gibco were cultured in complete Med-200 containing 1×LVES media supplement, while HUVEC cells from Lonza were cultured in EGM-2 medium with kit supplements added. All cells were cultured at 37° C. in 5% CO₂, routinely screened for mycoplasma, and authenticated by short tandem repeat DNA profiling performed by the University of Arizona Genetics Core Facility and were used for fewer than 50 passages. When appropriate, cells lines were cultured in a hypoxic environment (1% O₂, 5% CO₂, 94% N₂) using an InVivo2 400 hypoxia workstation (Baker Ruskinn).

### Kinase Assays

In vitro: Recombinant full-length GST-PIM1 was incubated with recombinant GST-HIF-1α, GST (negative control), or dephosphorylated myelin basic protein (MBP) in the presence or absence of AZD1208 (100 nM) and ³²P-labeled ATP in kinase assay buffer (20 mM MOPS [pH 7.0] containing 100 mM NaCl, 10 mmol/L MgCl₂, and 2 mmol/L dithiothreitol). Reactions were incubated at 37° C. for 60 min prior to the addition of 5× Laemmli sample buffer. Samples were separated by SDS-PAGE and gels were stained with Coomassie prior to autoradiography or preparation for mass spectrometry.

In vivo: HA-HIF-1α or its mutant (T455A) were immunoprecipitated with anti-HA antibodies. Immune complexes were washed three times in lysis buffer, then washed twice in 1× kinase buffer and incubated with 0.1 μg of recombinant active PIM1 and 100 μmol/L of ATP for 30 min at 25° C. Reactions were stopped by washing twice in a cold kinase buffer and boiling in 2×SDS loading buffer. The sample was separated on a SDS-polyacrylamide gel and subjected to Western blot analysis with anti-phospho HIF-1α (T455) antibody.

### DCE-MRI Studies

Two million PC3-VEC or PC3-PIM1 cells were injected subcutaneously into the rear flanks of mice and tumor volume was measured by caliper. Tumors were allowed to grow to ˜300 mm³ before initiating MRI studies using a 7T Bruker Biospec MRI instrument. Prior to the MRI scan, each mouse had a 27 G catheter placed in the tail vein and was anesthetized with 1.5-2.5% isoflurane in O₂ carrier gas. Physiologic respiration rate and core body temperature were monitored throughout the MRI session. All animals were imaged while maintaining their temperature at 37.0±0.2° C. using warm air controlled by a temperature feedback system (SA Instruments). The T1 relaxation time of each tissue of interest (30) was measured by acquiring a series of spin-echo MR images, variable TR and a RARE protocol using the following parameters: TR=150, 300, 350, 500, 700, 900, 1200, 2000, 3000 and 6000 msec; TE=9.07 msec; NEX=1; RARE factor=2; slice thickness=1.0 mm; FOV=2.0 cm2; linear encoding order; matrix=128×128; in-plane spatial resolution=0.23 mm2; hermite excitation pulse=90° for 2.7 msec duration with 2700 Hz bandwidth; and hermite refocusing pulse=180° for 1.71 msec duration with 2000 Hz bandwidth. The T1 time for each image voxel was estimated by non-linear regression of the variable TR signal to the following equation: (1) MZ (t)=MZ (1−e−TR/T1). A series of dynamic images were acquired using a Spoiled Gradient-echo MRI protocol with the following parameters: TR=50 msec; TE=8.07 msec; NEX=1; excitation pulse=158.9°; FOV=2.0 cm2; in-plane spatial resolution=0.23 mm2; matrix=128×128; slice thickness=1.0 mm, for a single slice centered in the tumor. Each image set was acquired in 6.4 sec and repeated 150 times for a total acquisition time of 16 min. An initial set of baseline images were acquired for 30 sec. prior to I.V. injection of 50 μL of MultiHance (Bracco Diagnostics Inc.) over a minute, which corresponds to a dose of 0.40 mMKg-1 for a 20 g mouse. All images were analyzed using the linear reference region model (Eq. [1]) after transforming the MRI signals to concentrations. Muscle in the left thigh of each mouse was used as the reference region. Upon sacrifice, tissue was harvested and stained by IHC for PIM1 and CD31.

ΔR1,TOI(T)=RKtrans·ΔR1,RR(T)+Ktrans,TOI/e,RR·0∫TΔR1,RR(t)dt−kep,TOI·0∫TΔR1,TOI(t)dt (1)

### In Vivo Studies

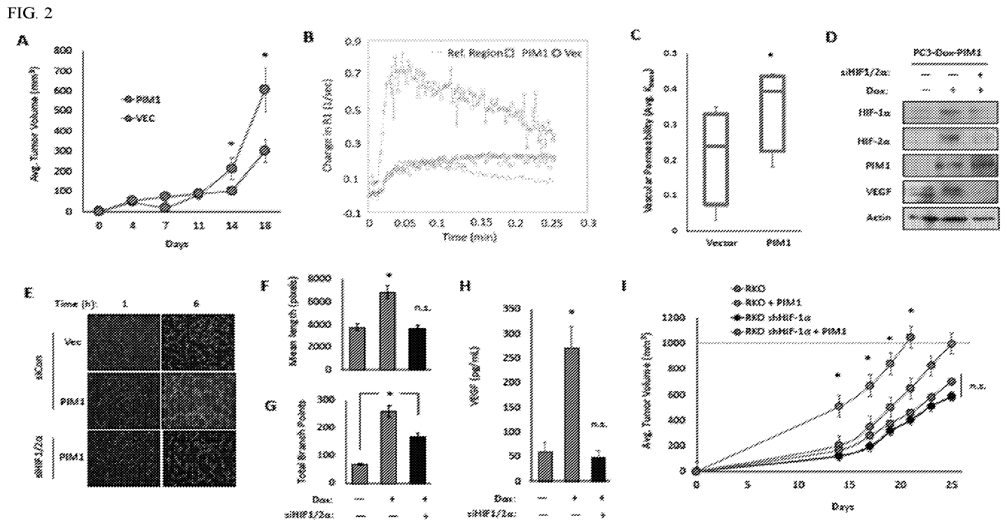

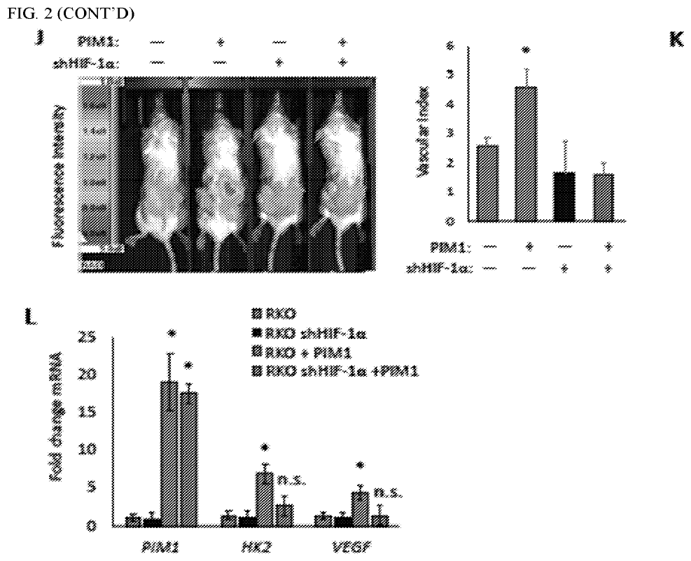

FIG. 2: Five million parental or HIF knockdown or PIM1 overexpressing or both RKO cells in PBS were injected subcutaneously into the rear flanks of mice. Tumor volume was measured overtime by caliper. At day 20, mice were injected with 2 nmol of Angiosense 750EX via tail vein injection. Twenty-four hours later, mice were anesthetized and imaged in a Lago Bioluminescence imager (Spectral Instruments). Mice were sacrificed when tumor burden (1000 mm³) or day 24 of the study. Tumors were harvested for mRNA analysis and IHC staining with HIF-1α, CC3, and CD3.

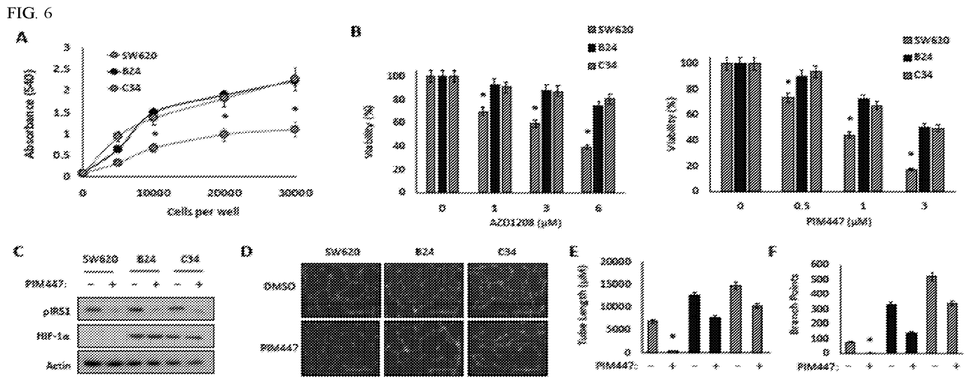

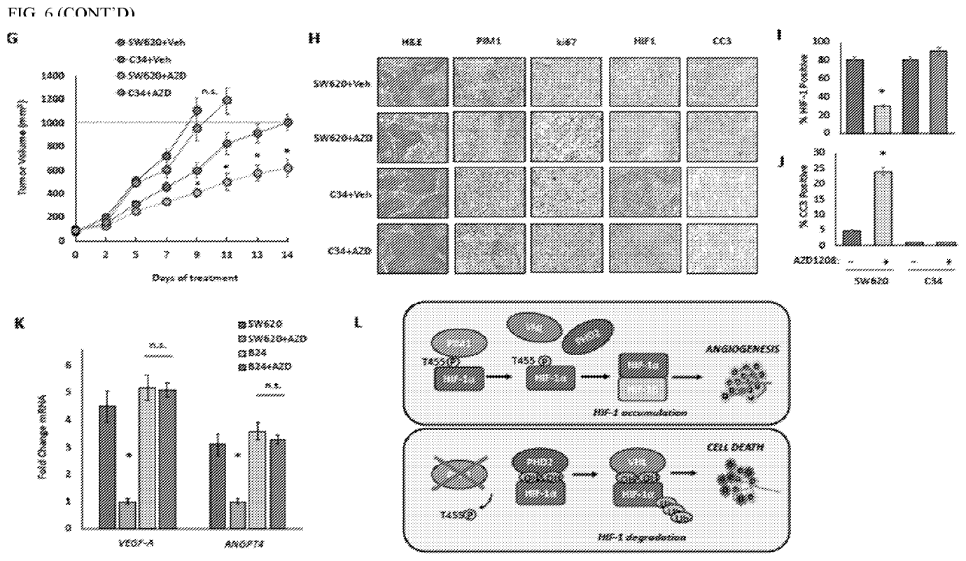

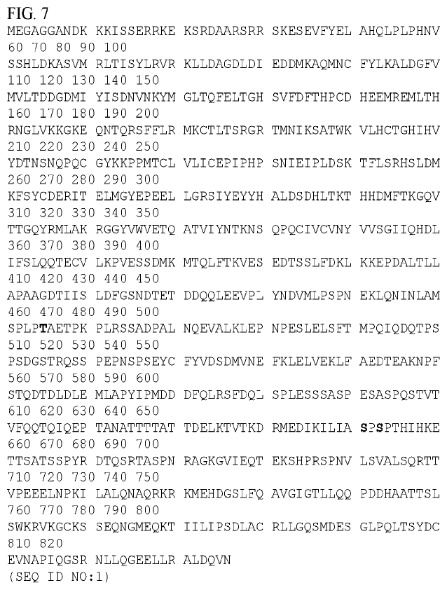

FIG. 6: Five million parental, B24, or C34 SW620 cells in PBS were injected subcutaneously into the rear flanks of mice. Once the tumors reached a volume of approximately 100 mm³, the mice were randomized for daily treatment with vehicle or AZD1208 (30 mg/kg/day, p.o.) for up to 2 weeks or until tumor burden reached a maximum volume of 2 cm³. Tumors were measured every 2-3 days via caliper. Upon sacrifice, tumors were harvested for IHC staining with PIM1, HIF-1α, and CC3, and mRNA was collected for analysis.

For both, the following formula was used to calculate tumor volume by caliper measurements:

V=(tumor width)²×tumor length/2.

### HUVEC Assay

Ten thousand HUVEC cells were plated onto 50 μL Geltrex matrix with 100 μL CM. CM from cancer cells was collected by adding 2 mL of DMEM or RPMI+0.5% FBS to 50,000 cells for 48 h before collection. Tubes were allowed to form for up to 6 h (Lonza kit) or 24 hours (Gibco kit) and then were stained with calcein AM and analysis was performed using ImageJ.

### Protein Extraction and Immunoblotting

Cultured cells were trypsinized and lysed in NP-40 lysis buffer (150 mM sodium chloride, 1% NP-40, 50 mM Tris pH 8.) with protease and phosphatase inhibitors. Proteins were separated via SDS-PAGE, transferred to PVDF membrane, and probed using the indicated antibodies.

### ELISA

CM from cancer cells was collected by adding 2 mL of DMEM+0.5% FBS to 50,000 cells for 48 hours before collection. Media was spun down to pellet cell debris and sterile-filtered before enzyme-linked immunosorbent assay (ELISA). ELISA was performed according to manufacturer's procedure (Novex by Life Technologies) and plates were read at 450 nm.

### MTT

Growth: Ten, twenty and thirty thousand cells were plated onto a 96-well plate and allowed to grow for 48 h before MTT assay.

Response to drug: Twenty thousand cells were plated onto a 96-well plate and allowed to grow for 24 h prior to addition of the drug, and MTT assays were performed after 24 h incubation with the indicated drugs. For MTT assays, media was removed from the cells by aspiration and 50 μL serum-free DMEM and 50 μL MTT solution were added to each well, and plates were incubated for 4 h at 37° C. After incubation, 150 μL of DMSO was added to each well for 15 minutes and absorbance was read at 540 nM.

qRT-PCR

Hypoxia-responsive gene expression was measured and quantified using RT² hypoxia-signaling PCR profiler arrays using the manufacturer's software (Qiagen). All other qRT-PCR reactions were performed using qPCRBIO SyGreen Blue Mix (PCR Biosystems), according to the manufacturer's protocol. Validated primer sets (QuantiTech primer assays; Qiagen) for each of the following genes were purchased to measure gene expression: VEGF-A, angiopoietin like 4 (ANGPTL4), and hexokinase 2 (HK2). HIF1α, PIM1 and Actin primers were purchased from IDT. Actin was used to normalize.

### Quantification and Statistical Analysis

All in vitro experiments, including HUVEC assays, western blots, MTT assays and RT-PCR analysis were conducted at least 3 independent times. T-test and linear regression analysis was used to compare differences between two groups. Two-way ANOVA was used to analyze differences among groups with multiple independent variables. Tumor growth was analyzed by fitting a mixed linear model of tumor volume vs. time for each mouse. All data are presented as the mean±SE, and a P<0.05 was considered to be statistically significant. Microsoft Excel and STATA15 were used for analyses.

### Mass Spectrometry

In-gel digestion. Gel slices were in-gel digested with trypsin (Pierce Biotechnology, Rockford, Ill.) for 3 h at 37° C. using ProteaseMax™ Surfactant trypsin enhancer following reduction and alkylation with dithiothreitol and iodoacetamide, respectively, according to the manufacturer's instructions (Promega Corporation, Madison, Wis.).

Mass spectrometry and database search were performed as previously described (Downs et al., 2018 J Proteomics 177, 11-20). Briefly, LC-MS/MS analysis was carried out using a Q Exactive Plus mass spectrometer (Thermo Fisher Scientific) equipped with a nanoESI source. Peptides were eluted from an Acclaim Pepmap™ 100 precolumn (100-μm ID×2 cm, Thermo Fischer Scientific) onto an Acclaim PepMap™ RSLC analytical column (75-μm ID×15 cm, Thermo Fischer Scientific) using a 5% hold of solvent B (acetonitrile, 0.1% formic acid) for 15 minutes, followed by a 5-22% gradient of solvent B over 105 minutes, 22-32% solvent B over 15 minutes, 32-95% of solvent B over 10 minutes, 95% hold of solvent B for 10 minutes, and finally a return to 5% of solvent B in 0.1 minutes and another 14.9 minute hold of solvent B. All flow rates were 300 nL/min using a Dionex Ultimate 3000 RSLCnano System (Thermo Fischer Scientific). Solvent A consisted of water and 0.1% formic acid. Data dependent scanning was performed by the Xcalibur v 4.0.27.19 software using a survey scan at 70,000 resolution scanning mass/charge (m/z) 400-1600 at an automatic gain control (AGC) target of 3e6 and a maximum injection time (IT) of 100 msec, followed by higher-energy collisional dissociation (HCD) tandem mass spectrometry (MS/MS) at 27nce (normalized collision energy), of the 10 most intense ions at a resolution of 17,500, an isolation width of 1.5 m/z, an AGC of 2e5 and a maximum IT of 50 msec. Dynamic exclusion was set to place any selected m/z on an exclusion list for 20 seconds after a single MS/MS. Ions of charge state+1, 7, 8, >8 and unassigned were excluded from MS/MS, as were isotopes. Tandem mass spectra were extracted from Xcalibur ‘RAW’ files and charge states were assigned using the ProteoWizard 3.0 msConvert script using the default parameters. The fragment mass spectra were then searched against the human SwissProt_2018 database (20413 entries) using Mascot (Matrix Science, London, UK; version 2.6.0) using the default probability cut-off score. The search variables that were used were: 10 ppm mass tolerance for precursor ion masses and 0.5 Da for product ion masses; digestion with trypsin; a maximum of two missed tryptic cleavages; variable modifications of oxidation of methionine and phosphorylation of serine, threonine, and tyrosine. Cross-correlation of Mascot search results with X! Tandem was accomplished with Scaffold (version Scaffold_4.8.2; Proteome Software, Portland, Oreg., USA). Probability assessment of peptide assignments and protein identifications were made through the use of Scaffold. Only peptides with ≥95% probability were considered.

### Example 1

PIM1 Expression is Correlated with Angiogenesis in Human Cancers

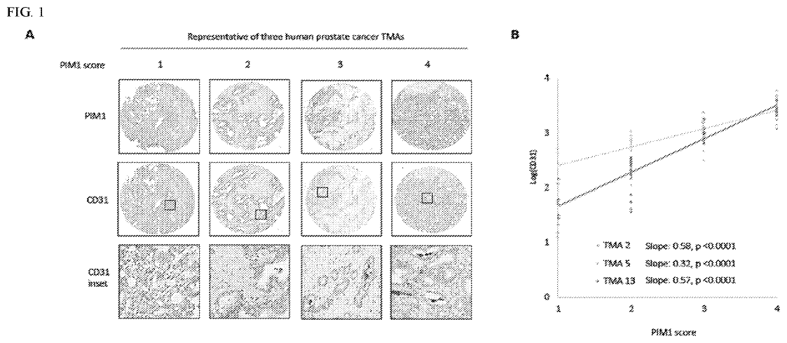

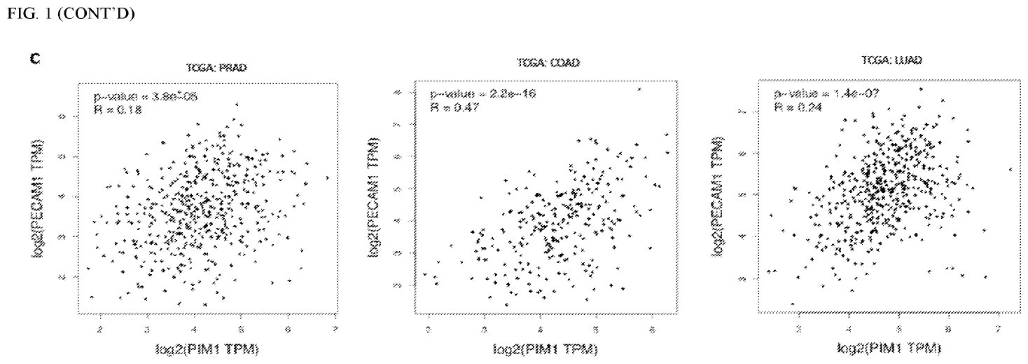

Tumor vasculature is responsible for providing the nutrients and oxygen required for survival and proliferation, as well as a route for dissemination. It was recently learned that overexpression of PIM1 kinase was sufficient to sustain vasculature during treatment with anti-VEGF therapy. Therefore, it was determined whether PIM1 expression was correlated with tumor angiogenesis. To this end, immunohistochemistry (IHC) was performed to quantify PIM1 and CD31 (an endothelial cell marker) levels in three prostate cancer tissue microarrays (TMAs) (#2 n=36, #5 n=44, and #13 n=29). These TMAs were obtained from diagnostic cores of radical prostatectomies of patients prior to treatment at the University of Arizona, and samples ranged in severity of disease (Gleason score 6-9). A statistically significant correlation was observed between PIM1 expression and microvessel density (FIGS. 1A and B). To provide further evidence for the association between PIM1 and angiogenesis, the correlation between PIM1 and PECAM1 transcript levels was investigated in publicly available data from The Cancer Genome Atlas database. Prostate, colon, and lung cancer all displayed a statistically significant correlation between PIM1 and PECAM1 (FIG. 1C). Moreover, PIM1 and VEGF-A transcript levels were significantly correlated in prostate, colon, and lung cancer. Thus, PIM1 expression is significantly correlated with vascularization in human tumor samples.

### Example 2

### PIM1 Induces Angiogenesis in a HIF-1-Dependent Manner

With the correlative relationship between PIM1 and tumor vasculature established in human tumors, it was next determined whether PIM1 is sufficient to promote angiogenesis in vivo using pre-clinical imaging modalities to quantitatively measure changes in vascular perfusion over time. To this end, 1×10⁶ PC3 prostate cancer cells were stably transfected with PIM1 or a vector control (VEC) and implanted subcutaneously into the flanks of male NSG mice. Verifying previous results, PIM1-expressing tumors grew significantly faster than wild-type tumors (FIG. 2A). To assess angiogenesis, a paramagnetic contrast agent (CA) was injected intravenously, and magnetic resonance imaging (MRI) was performed to visualize uptake of the CA in the tumor and reference tissue and generate a time-concentration curve. The resulting curve was fit to a pharmacokinetic model to estimate physiological parameters for the tissue of interest. As the CA passes through the circulation (typically 45-60 s after injection), it is predominantly intravascular, allowing for the evaluation of perfusion (i.e., blood flow per unit volume). During the subsequent 2-15 min, the contrast agent passes into the extravascular space, allowing for measurement of vascular permeability (Rₖₜᵣₐₙₛ) and relative clearance rate. Tumors were imaged once they reached approximately 300 mm³, and each cohort was imaged again 7 days later. To compare angiogenesis, DCE-MRI data from size-matched PC3/VEC and PC3/PIM1 tumors were analyzed using the Linear Reference Region Model, which has superior resolution to current methods (Cardenas-Rodriguez et al., 2013 Magnetic resonance imaging 31, 497-507). The resulting time-concentration curve revealed a dramatic increase in the uptake of the CA in PIM1-expressing tumors compared to control tumors, indicating that PIM1 expression significantly increases perfusion (FIG. 2B). Furthermore, the average vascular permeability (Rₖₜᵣₐₙₛ) was significantly greater in PIM1-overexpressing tumors than in control tumors (FIG. 2C). Taken together, these analyses indicate that PIM1-overexpressing tumors have more blood flow and a greater number of mature vessels compared to VEC. This correlation was confirmed with endpoint staining of CD31, which demonstrated that microvessel density was significantly higher in PIM1 tumors than in control tumors. Taken together, these data demonstrate that PIM1 expression is sufficient to enhance tumor angiogenesis.

HIF-1 is a transcription factor that is a master regulator of angiogenesis, and we previously reported that PIM inhibitors can reduce the levels of HIF-1α. Therefore, it was determined whether the pro-angiogenic effect of PIM1 is dependent on HIF-1. To determine whether HIF-1 activation is required for the pro-angiogenic effect of PIM1, HIF-1/2α were knocked down using siRNA in PC3 cells stably expressing a doxycycline (Dox)-inducible lentiviral vector encoding PIM1 (Dox-PIM1) (Zhang et al., 2018 Cancer Sci 109, 1468-1479), and in vitro angiogenesis assays were performed. Forty-eight hours after knockdown, Dox-PIM1 cells were treated with Dox, and cell lysates and conditioned media (CM) were collected after 24 h. Then, human umbilical vein endothelial cells (HUVECs) were suspended in CM from each experimental condition, plated on reduced growth factor basement membrane, and tube formation was assessed over time. Immunoblotting verified that PIM1 stabilized HIF-1/2α, and siRNA effectively reduced HIF-1/2α levels (FIG. 2D). Fluorescence images of calcein AM-stained endothelial cells were acquired over 6 h, which corresponded with maximal tube formation (FIG. 2E). CM from Dox-PIM1 cells substantially enhanced tube formation compared to CM from Dox-VEC cells. Strikingly, the pro-angiogenic effect of PIM1 expression was abolished in cells lacking HIF-1/2α (FIG. 2E). Image analysis verified that PIM1 overexpression significantly increased the average tube length and number of branch points, and HIF-1/2α knockdown restored tube formation to basal levels (FIGS. 2F and G). To confirm that PIM1 expression impacts the release of pro-angiogenic factors from tumor cells through the upregulation of HIF-1 target genes, the amount of vascular endothelial growth factor (VEGF) was measured in the CM from each sample. Conditioned media from PIM1-expressing cells contained nearly 5-fold more VEGF than control cells, and knockdown of HIF-1/2α restored VEGF levels to basal levels (FIG. 2H). RKO colon cancer cell lines stably overexpressing PIM1 or a vector control in combination with stable knockdown of HIF-1α were generated. In vitro angiogenesis assays using CM from each cell line demonstrated that PIM1 expression significantly increased mean tube length and total branch points compared to vector control, whereas PIM1 was unable to induce tube formation in RKO shHIF-1α cells. To show that the pro-angiogenic effect of PIM1 requires HIF-1α in vivo, 5×10⁶ RKO cells were injected subcutaneously into the flanks of SCID mice, and tumor growth was measured over time and angiogenesis was assessed at several time points by in vivo imaging. As expected, PIM1-overexpressing tumors grew significantly faster than controls (FIG. 2I). Knockdown of HIF-1α abolished the growth advantage of PIM1 overexpression (FIG. 2I). To monitor angiogenesis, mice from each group were injected with Angiosense 750EX, a fluorescent probe that remains localized in the vasculature to allow for in vivo imaging of angiogenesis. To calculate the relative amount of functional vasculature in each cohort, Angiosense signal was normalized to differences in tumor volume to obtain a vascular index for each group. RKO tumors expressing PIM1 were significantly more vascular than the parental line, whereas no increase in angiogenesis was observed with PIM1 overexpression in tumors lacking HIF-1α (FIG. 2J). CD31 staining of tumors from each group confirmed that the pro-angiogenic effect of PIM1 was lost in tumors with knockdown of HIF-1α (FIG. 2K). Taken together, these data indicate that expression of PIM1 promotes angiogenesis in a HIF-1-dependent manner and suggests that the pro-tumorigenic effect of PIM1 can largely be attributed to its ability to promote angiogenesis.

### Example 3

### PIM1 Promotes Pro-Angiogenic Gene Expression Through HIF-1

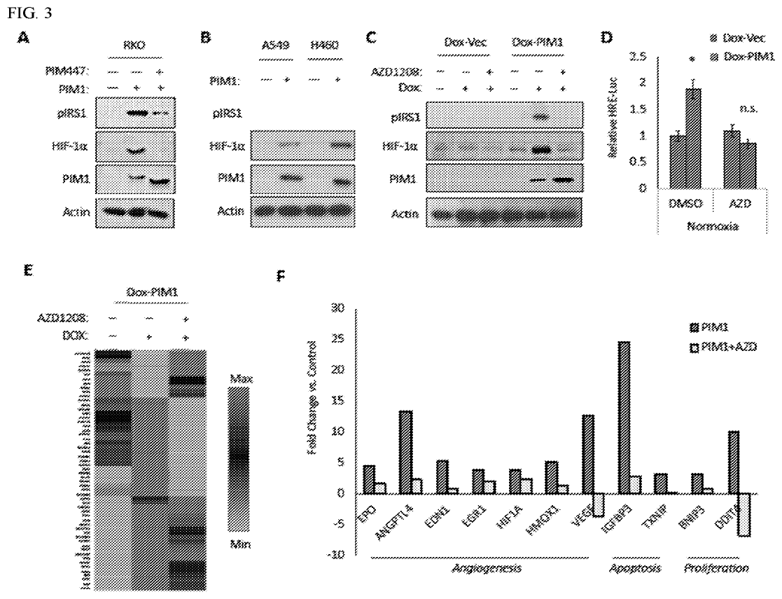

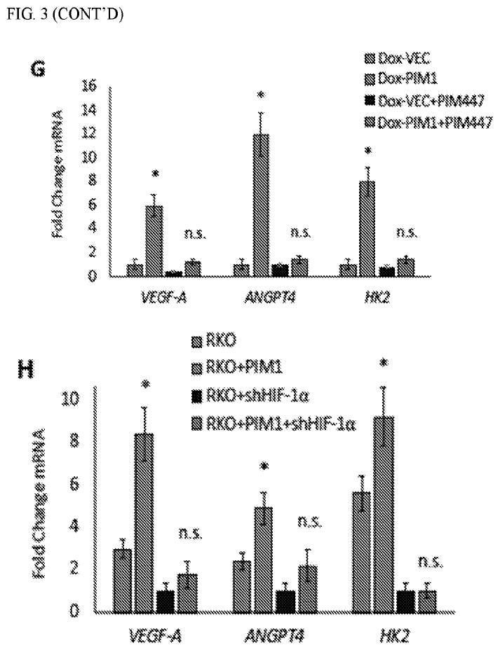

Because the pro-angiogenic effect of PIM1 is dependent on HIF-1α, it was hypothesized that elevated PIM1 expression is sufficient to activate HIF-1 in the absence of hypoxia. Immunoblotting was used to evaluate HIF-1α protein levels in control and PIM1-overexpressing colon (RKO), prostate (PC3), and lung (A549) cell lines. Strikingly, PIM1 expression was sufficient to stabilize HIF-1α in all cell lines tested (FIG. 3A-C). Importantly, treatment with chemically distinct pan-PIM kinase inhibitors (PIM447 and AZD1208) blocked the ability of PIM1 to increase HIF-1α protein levels (FIGS. 3A and C). To ensure that the levels of HIF-1α observed after PIM1 induction were sufficient to activate HIF-dependent transcription, Dox-VEC or Dox-PIM1 cells were co-transfected with Renilla-luciferase and a previously described HIF-1 reporter that drives luciferase expression (HRE-Luc) (Rapisarda et al., 2002 Cancer research 62, 4316-4324), treated for 24 h with Dox to induce PIM1, and then treated with DMSO or AZD1208 for 4 h. To account for increased cell growth and death due to PIM1 overexpression and PIM inhibitor treatment, respectively, the HRE-Luc signal was normalized to Renilla-Luc levels. PIM1 expression increased HIF-1 activity by approximately 2-fold in normoxia, and this effect was reversed by treatment with the PIM inhibitor (FIG. 3D). To assess the effect of PIM1 on HIF-1 target genes, a semi-high throughput qPCR assay was used to measure a panel of 84 hypoxia-inducible genes (Qiagen RT Profiler). PC3 Dox-PIM1 cells were cultured in normoxic conditions with or without 20 ng/mL Dox for 24 h to induce PIM1 and treated±AZD1208 for 8 h, at which point mRNA was collected for subsequent gene expression analysis. PIM1 expression altered the transcript levels of several classes of hypoxia-responsive genes, including critical mediators of angiogenesis, proliferation, and apoptosis (FIG. 3E). To identify genes whose induction was specific to PIM1, genes that were unregulated at least 3-fold by PIM1 expression in normoxia and significantly reduced by treatment with AZD1208 were analyzed. Of the eleven genes that fit these criteria, seven are known to promote angiogenesis, and all are established targets of HIF-1 (FIG. 3F). It was validated that PIM1 increased the expression of several well-known HIF-1 target genes (VEGF-A, ANGPT4, and HK2) by qRT-PCR, and treatment with PIM447 restored the expression of each to basal levels (FIG. 3G). To confirm that PIM1 alters gene expression in a HIF-1-dependent manner, the expression of the same set of HIF-1 target genes in the previously described RKO cell line with stable knockdown of HIF-1α was analyzed. PIM1 overexpression significantly increased the transcript level of all three genes compared to control cells, whereas no increase was observed in RKO cells lacking HIF-1α (FIG. 3H). Thus, PIM1 expression is sufficient to stabilize HIF-1/2α in normoxic conditions and increase the transcription of HIF-1 target genes.

### Example 4

### PIM1 Phosphorylates HIF-1α at Threonine 455

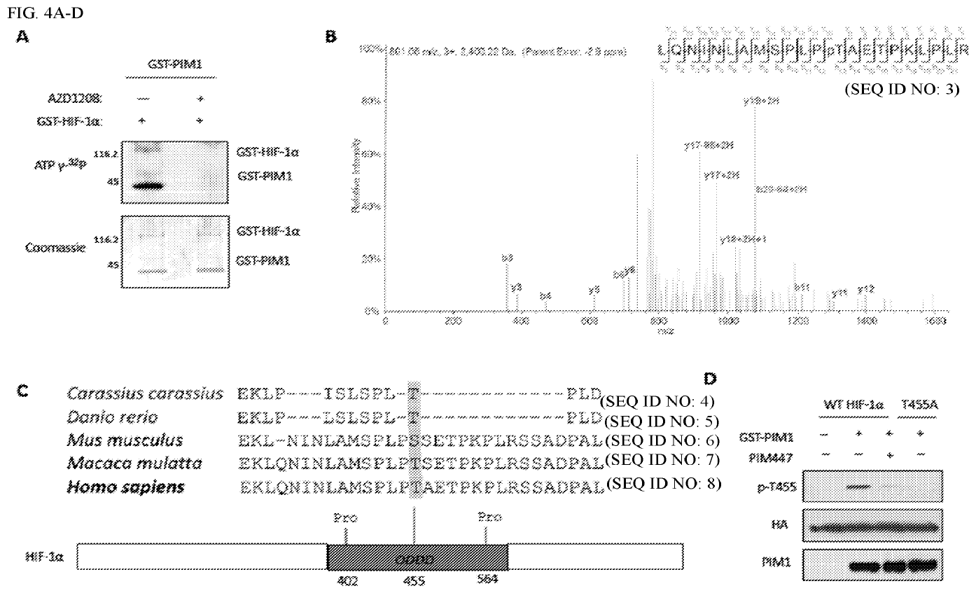

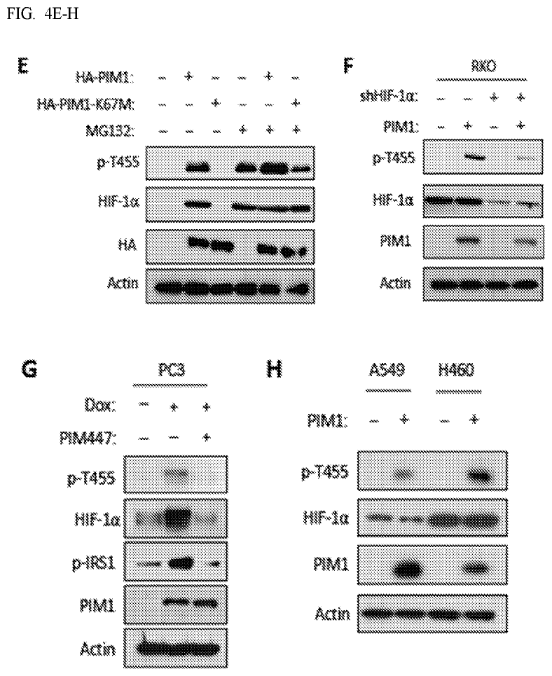

Next, the mechanism by which PIM1 stabilizes HIF-1α was determined. Because PIM1 is a serine-threonine kinase, it was hypothesized that PIM1 may directly phosphorylate HIF-1α to alter its protein stability. To test this, in vitro kinase assays were performed using recombinant PIM1 and HIF-1α. Autoradiography revealed that PIM1 is able to phosphorylate HIF-1α, and phosphorylation was lost in the presence of a PIM inhibitor (FIG. 4A). To identify PIM-mediated phosphorylation sites, HIF-1α was isolated and mass spectrometry analysis was performed to identify post-translational modifications. PIM1 phosphorylated HIF-1α at two sites in vitro. The first, Ser643, has been previously described as an ERK target site that enhances the nuclear localization of HIF-1α but does not alter its protein stability (Mylonis et al., 2006 J Biol Chem 281, 33095-33106). The second site, Thr455, is a previously uncharacterized site located within the ODDD of HIF-1α between Pro402 and Pro564, which are hydroxylated by PHDs as a signal initiating the proteasomal degradation of HIF-1α (FIG. 4B). Notably, Thr455 is evolutionarily conserved among mammals, demonstrating its importance as a regulatory site (FIG. 4C). Based on its localization within the ODDD, the effect of Thr455 phosphorylation on HIF-1α stability was investigated. To verify that this site is phosphorylated in cells, PIM1 and HA-HIF-1α were co-transfected into 293T cells, HIF-1α was immunoprecipitated, and mass spectrometry was used to detect post-translational modifications. HIF-1α was robustly phosphorylated at Thr455 in cells expressing PIM1 (FIG. S3). Next, we generated a phospho-specific antibody against HIF-1α Thr455. To verify the specificity of this antibody, site-directed mutagenesis was used to create a T455A mutant of HIF-1α. Wild-type or T455A HA-HIF-1α were immunoprecipitated from cells and incubated with recombinant PIM1 in the same conditions used for in vitro kinase assays. Wild-type HIF-1α displayed robust phosphorylation at Thr455 by PIM1, which was blocked by AZD1208, whereas the T455A mutant was not recognized by the phospho-antibody (FIG. 4D). These results confirm that PIM1 directly phosphorylates HIF-1α at T455 and the antibody specifically recognizes phosphorylation at this site. To further confirm that PIM1 phosphorylates HIF-1α in cells, HEK293T cells were transfected with vector, HA-PIM1, or a kinase-dead PIM1 (K67M), and total and phospho-HIF-1α (T455) were assessed by immunoblotting. At the protein level, HIF-1α was only upregulated and phosphorylated in cells expressing kinase-active PIM1 (FIG. 4E). Alternatively, almost no Thr455 phosphorylation was observed in cells expressing kinase-dead PIM1, indicating that this construct acts as a dominant negative (FIG. 4E). To confirm that Thr455 phosphorylation in PIM1-expressing cells was not solely due to the increased abundance of HIF-1α, 293T cells were treated with a proteasome inhibitor (MG-132) for 4 h to stabilize HIF-1α in normoxia. In this context, phosphorylation of Thr455 was observed in basal conditions and significantly increased upon overexpression of PIM1 (FIG. 4E). Next, PIM expression and activity was altered in several cancer cell lines to ensure that Thr455 phosphorylation was universally observed in cancer cell lines in which we established that PIM1 increases HIF-1α (FIG. 3A-C). PIM1 overexpression increased Thr455 phosphorylation in RKO colon cancer cells, and this effect was reduced upon knockdown of HIF-1α, demonstrating the specificity of this antibody (FIG. 4F). Similarly, induction of PIM1 in PC3 Dox-PIM1 cells increased Thr455 phosphorylation, and co-treatment with PIM447 blocked the induction of phospho-T455 and total HIF-1α (FIG. 4G). PIM1 overexpression also induced Thr455 phosphorylation in the lung cancer cell lines A549 and H460 (FIG. 4H). Taken together, these studies establish that PIM1 directly phosphorylates HIF-1α at Thr455.

### Example 5

### PIM-Mediated Phosphorylation of HIF-1α at Thr455 Increases its Protein Stability

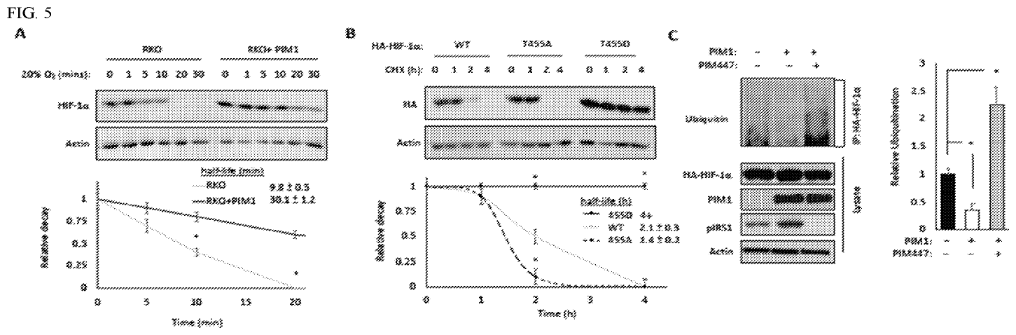

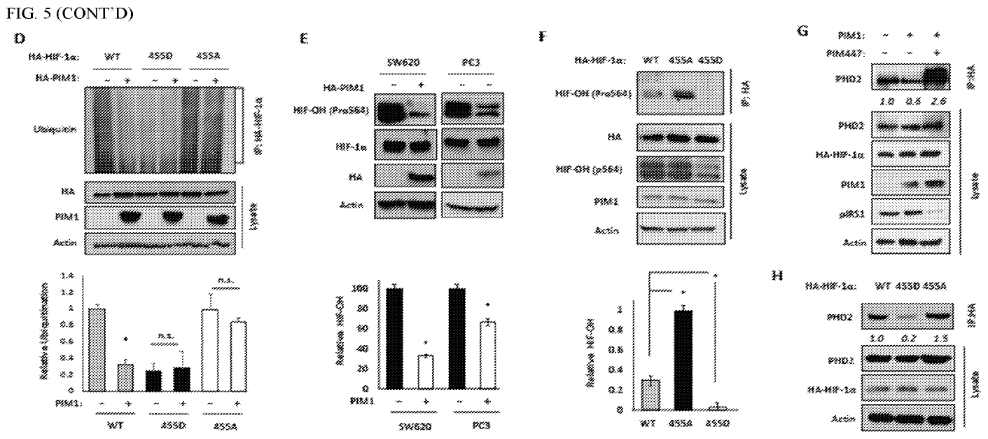

Next, the effect of Thr455 phosphorylation on HIF-1α protein levels was characterized. Wild-type and RKO cells stably expressing PIM1 were cultured in hypoxia (1% O₂) for 4 hours to stabilize HIF-1α and then returned to normoxia (20% O₂), and lysates were collected over a 30-min time course. The half-life of HIF-1α was significantly longer in PIM1-expressing cells than in wild-type cells (30.1±1.2 vs. 9.8±0.5 mins) (FIG. 5A). To directly assess the effect of Thr455 phosphorylation on HIF-1α protein stability, site-directed mutagenesis was used to generate HIF-1α T455D (phosphomimetic) and T455A (phospho-null) constructs. Following transfection of WT, T455D, or T455A HIF-1α, HEK293T cells were treated with cycloheximide and lysates were collected over time to determine the rate of protein degradation. The half-life of the phospho-null mutant (T455A) was significantly shorter than that of WT HIF-1α (1.4±0.2 vs. 2.1±0.2 h), whereas the half-life of the phospho-mimetic (T455D) was significantly longer, showing little degradation over the 4-h time course (FIG. 5B). Because HIF-1α is primarily degraded by the proteasome in normoxic conditions, it was tested whether PIM1 decreased HIF-1α ubiquitination. HEK293T cells stably expressing PIM1 or a vector control were transfected with HA-HIF-1a and treated with DMSO or PIM447 for 30 min, followed by MG-132 treatment for 4 h to allow for the accumulation of ubiquitinated HIF-1α. HIF-1α was immunoprecipitated and ubiquitin was detected by immunoblotting. PIM1 expression decreased the amount of ubiquitin bound to HIF-1α by approximately 3-fold, whereas PIM inhibition significantly increased the amount of ubiquitination by over 2-fold (FIG. 5C). To directly assess the effect of Thr455 phosphorylation on ubiquitination, 293T cells expressing VEC or PIM1 were transfected with HA-HIF-1α WT, T455D, or T455A, treated with MG-132 for 4 hours, and HA-HIF-1α was immunoprecipitated. Overexpression of PIM1 significantly decreased WT HIF-1α ubiquitination compared to controls, whereas PIM1 expression did not alter the ubiquitination of T455D and T455A HA-HIF-1α (FIG. 5D). Moreover, the level of ubiquitination of T455A was similar to that of wild-type HIF-1α, whereas T455D ubiquitination was significantly lower than that of wild-type HIF-1a and similar to the amount of ubiquitination observed for wild-type HIF-1α with PIM1 overexpression (FIG. 5D).

Because hydroxylation is the initiating step in the canonical HIF-1α degradation pathway, changes in HIF-1α hydroxylation at Pro564 were assessed. Hydroxylation of HIF-1α at Pro564 was significantly reduced in SW620 colon and PC3 prostate cancer cells overexpressing PIM1 (FIG. 5E). In a parallel experiment, HA-HIF-1α WT, T455D, or T455A were immunoprecipitated from 293T cells stably expressing VEC or PIM1 (to exclude any endogenous HIF-1α) after 4 h treatment with MG-132 to allow for the accumulation of hydroxylated HIF-1α. The T455D mutant showed no visible hydroxylation, whereas hydroxylation of the T455A mutant was significantly increased compared to WT HIF-1α (FIG. 5F). PHD2 is the primary isoform responsible for hydroxylating HIF-1α. Because Thr455 is located within the ODDD, it was hypothesized that phosphorylation at this site may disrupt PHD binding to HIF-1α. To this end, HA-HIF-1α was transfected into 293T cells stably expressing PIM1 or vector control. Cells were treated with MG132 for 4 h, HA-HIF-1α was immunoprecipitated, and PHD2 binding was assessed by immunoblotting. Significantly less PHD2 was bound to HIF-1α in cells overexpressing PIM1 compared to VEC cells (FIG. 5G). To test whether Thr455 phosphorylation alters PHD2 binding, 293T cells were transfected with HA-HIF-1α WT, T455D, or T455A constructs and treated with MG-132 for 4 h prior to immunoprecipitation of HA-HIF-1α variants. Significantly more PHD2 was bound to HIF-1α T455A than wild-type, whereas significantly less was bound to HIF-1α T455D compared to wild-type (FIG. 5H). These data indicate that PIM1-mediated phosphorylation of HIF-1α at Thr455 increases protein stability by blocking PHD binding, hydroxylation, and subsequent proteasomal degradation of HIF-1α.

### Example 6

### The Anti-Tumor Effects of PIM Inhibitors Depend on Downregulation of HIF-1

To confirm the significance of Thr455 phosphorylation in vivo and in vitro, two homozygous HIF-1α T455D mutant SW620 colon cancer cell lines were generated using CRISPR site-directed mutagenesis. These lines were validated by Sanger sequencing. Both of these cell lines showed stable HIF-1α protein in normoxic conditions and had significantly increased expression of HIF-1 target genes (FIG. 6C). To assess cell growth, parental SW620 and HIF-1α T455D mutant cell lines (B24 and C34) were plated at various densities (1000, 2000, and 3000 cells per well) and allowed to grow for 48 h. Then, MTT assays were performed to assess relative cell number. The HIF-1α T455D clones, B24 and C34, displayed significantly increased growth compared to parental SW620 cells (FIG. 6A). Next, cells were treated with increasing doses of AZD1208 and PIM447 for 24 h, and MTT assays were performed to assess cell viability. Parental SW620 cells exhibited a significant and dose-dependent reduction in viability in response to both PIM inhibitors, whereas the B24 and C34 cell lines were less sensitive (FIG. 6B). To confirm that HIF-1α was refractory to PIM inhibition in the T455D mutant cell lines, cells were treated with PIM447 (1 μM) for 4 h. Immunoblotting revealed that PIM447 reduced pIRS1 (S1101), a known PIM substrate, whereas HIF-1α levels were significantly higher and refractory to PIM inhibition in the T455D mutant cell lines (FIG. 6C). To determine whether Thr455 phosphorylation also impacted angiogenesis, CM was harvested from parental and B24 and C34 SW620 cells treated with or without PIM447 for 24 h and used for in vitro tube formation assays. CM from both B24 and C34 cells significantly increased both the total tube length and number of branch points compared to parental SW620 CM. Treatment with PIM447 significantly reduced the tube length and number of branch points resulting from SW620 CM compared to DMSO, whereas the mutant cell lines were refractory to the anti-angiogenic effect of PIM inhibitors (FIG. 6D-F). Next, the tumorigenicity of these cell lines and sensitivity to PIM inhibition in vivo was assessed. Ten million parental SW620, C34, or B24 cells were injected into the flanks of SCID mice. Tumors were allowed to grow to an average size of 100 mm³, and then the mice were randomly segregated into vehicle or AZD1208 (30 mg/kg) treatment groups. Tumor growth was measured by caliper every other day, and tumors were harvested for mRNA and IHC analysis once maximum tumor burden was reached. Both B24 and C34 tumors grew significantly faster than parental SW620 tumors (FIG. 6G). Strikingly, treatment with AZD1208 significantly reduced the volume of SW620 tumors but was unable to slow the growth of either the B24 or C34 mutant xenografts (FIG. 6G). Hematoxylin and eosin (H&E) staining revealed that AZD1208 disrupted tumor vasculature in the SW620 xenografts, resulting in necrotic tissue, whereas regular vasculature was observed in C34 and B24 tumors, regardless of PIM inhibition (FIG. 6H). Importantly, AZD1208 significantly reduced HIF-1α levels and increased apoptosis (as assessed by cleaved caspase-3 staining) in SW620 tumors, whereas HIF-1α was refractory to PIM inhibition and no significant apoptosis was observed in B24 or C34 tumors (FIGS. 6I and J). RT-PCR analysis of tumor tissue confirmed that PIM inhibition reduced the expression of pro-angiogenic genes VEGF-A and ANGPT4 in SW620 tumors, whereas PIM inhibition had no effect on HIF-1 target genes in B24 and C34 tumors (FIG. 6K). These data indicate that phosphorylation of Thr455 is sufficient to drive angiogenesis and increase tumor growth. Moreover, the fact that a single point mutation in HIF-1a made these tumors largely refractory to PIM inhibition suggests that the anti-tumor effects of small molecule PIM inhibitors is primarily due to their ability to downregulate HIF-1 and reduce angiogenesis.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A composition comprising:
a monoclonal antibody that inhibits one or more activities of HIF-1α.

2. The composition of claim 1, wherein said monoclonal antibody binds to phosphorylated HIF-1α.

3. The composition of claim 1, wherein said monoclonal antibody blocks the phosphorylation of HIF-1α by PIM.

4. The composition of claim 3, wherein said PIM is PIM1, PIM2, or PIM3.

5. The composition of claim 2, wherein said phosphorylation is phosphorylation at Thr455 of HIF-1α.

6. The composition of claim 1, wherein said monoclonal antibody is humanized.

7. The composition of claim 1, wherein said monoclonal antibody human, chimeric, or murine.

8. The composition of claim 1, wherein said monoclonal antibody is an antibody fragment.

9. The composition of claim 1, wherein said composition is a pharmaceutical composition.

10. The composition of claim 1, wherein said composition further comprises a pharmaceutically acceptable carrier.

11. The composition of claim 9, wherein said composition further comprises an additional anti-cancer agent.

12. The composition of claim 11, wherein said anti-cancer agent is selected from the group consisting of a chemotherapeutic agent, anti-angiogenic agent, and a PIM kinase inhibitor.

13. The composition of claim 12, wherein said anti-angiogenic agent is selected from the group consisting of axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept.

14. The composition of claim 12, wherein said PIM kinase inhibitor is selected from the group consisting of AZD1208, LGH447, SGI-1776, PIM447, SEL24, and TP-3654.

15. A method of treating cancer, comprising:
administering the composition of claim 1 to a subject diagnosed with cancer, wherein said administering treats one or more signs or symptoms of cancer in said subject.

16. A method of treating cancer, comprising:
a) identifying the presence of phosphorylation at Thr455 of HIF-1α in a sample from a subject diagnosed with cancer; and
b) treating said subject with a PIM kinase inhibitor and/or an anti-angiogenic agent.

17. (canceled)

18. The method of claim 16, wherein said identifying comprises contacting said sample with a monoclonal antibody that specifically binds to said Thr455 of HIF-1α and detecting said binding.

19. The method of claim 14, wherein said cancer is a solid tumor.

20. The method of claim 19, wherein said cancer prostate, colon, breast, or lung cancer.

21-24. (canceled)


SEQUENCE LISTING (8 sequences)

SEQ ID NO: 1 - protein, 826 aa, Artificial sequence
  synthetic
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 2 - DNA, 83 bp, Artificial sequence
  synthetic
aaaattacagaatataaatttggcaatgtctccattacccgatgctgaaacgccaaagccacttcgaagtagtgctgaccctg

SEQ ID NO: 3 - protein, 22 aa, Artificial sequence
  synthetic
LQNINLAMSPLPTAETPKLPLR

SEQ ID NO: 4 - protein, 14 aa, Carassius carassius
EKLPISLSPLTPLD

SEQ ID NO: 5 - protein, 14 aa, Danio rerio
EKLPLSLSPLTPLD

SEQ ID NO: 6 - protein, 29 aa, Mus musculus
EKLNINLAMSPLPSSETPKPLRSSADPAL

SEQ ID NO: 7 - protein, 30 aa, Macaca mulatta
EKLQNINLAMSPLPTSETPKPLRSSADPAL

SEQ ID NO: 8 - protein, 30 aa, Homo sapiens
EKLQNINLAMSPLPTAETPKPLRSSADPAL